(19) United States
(12) Patent Application Publication
(10) Pub. No.: US 2006/0088560 A1
(43) Pub. Date: Apr. 27, 2006

(54) Cosmetic use of mangiferin

(75) Inventors: Laetitia Charrier, Paris (FR); Frederique Poirier, Clamart (FR); Georges Maillet, Malakoff (FR); Chrisitan Lubrano, Sevres (FR)

Correspondence Address: YOUNG & THOMPSON, 745 SOUTH 23RD STREET, 2ND FLOOR, ARLINGTON, VA, 22202, US

(73) Assignee: LABORATOIRES DE BIOLOGIE VEGETALE YVES ROCHER, LA GACILLY (FR)

(21) Appl. No.: 11/251,914

(22) Filed: Oct. 18, 2005

(30) Foreign Application Priority Data
Oct. 21, 2004 (FR) 04 11227

(51) Int. Cl. A61K 8/49

(52) U.S. Cl. 424/401

(57) ABSTRACT

The present invention concerns a cosmetic use of mangiferin, or one of its isomers or derivatives, in particular a plant extract containing mangiferin, for example a leaf extract of Aphloia or Mangifera, for preventing and/or reducing the effects caused by heat stress on the skin, lips or hair.

The present invention concerns cosmetic uses of mangiferin (or its derivatives), or of a plant extract containing mangiferin, in particular a leaf extract of Aphloia or Mangifera.

Mangiferin is a C-glucoside of tetrahydroxy-1,3,6,7 xanthone. It is also called aphloiol (Billet et al., 1965). This molecule and its isomers (isomangiferin) and derivatives (methylated, O-glycosyl derivatives) are naturally present in a certain number of plants, but it is mangiferin which is the most widely distributed xanthone C-glucoside (Hostettmann, 1977), and more particularly among Angiosperms.

Mangiferin has the following chemical structure:

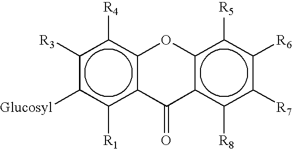

wherein R₁═R₃═R₆═R₇═OH and R₄═R₅═R₈═H.

Isomangiferin also exists naturally, having the following structure:

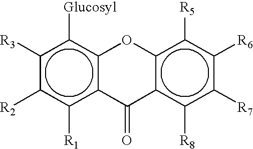

wherein R₁═R₃═R₆═R₇═OH and R₂═R₅═R₈═H.

Its derivatives of natural origin may have O-methyl groups (—OCH₃) or glucosyl groups (—C₆H₁₁O₆) at positions R₁, R₃, R₆ or R₇.

All the compounds formed by mangiferin, its isomers and its derivatives correspond to the following general formula I:

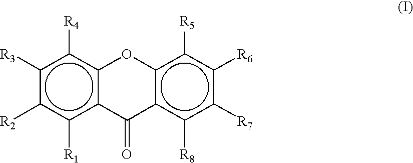

wherein R₁, R₂, R₃, R₄, R₅, R₆, R₇, R₈ are chosen from —H, —OH, —OCH₃ and a glucosyl radical.

Compounds of formula I may be obtained by different means:

1) Extraction from Plant Material.

Mangiferin, its isomers and its derivatives are naturally present in different plants, in particular in the species indicated in Table 1 below:

TABLE 1

| Class | Sub-Class | Order | Family | Species |
|---|---|---|---|---|
| Filices | Leptosporangiatae | Filicales | Polypodiaceae | Athyrium mesosorum |
| Dicotyledoneae | Archichlamydeae | Guttiferales | Guttiferae | Hypericum acutum |
| | | | | H. chinense |
| | | | | H. humifusum |
| | | | | H. montanum |
| | | | | H. nummularium |
| | | | | H. pulchrum |
| | | Rosales | Leguminosae | Hedysarum obscurum |
| | | Rutales | Malphighiaceae | Hiptage madablota |
| | | Sapindales | Anacardiaceae | Mangifera indica |
| | | Celastrales | Hippocrateaceae | Salacea prunoides |
| | | Violales | Flacourtiaceae | Flacourtia indica |
| | | | | Aphloia theaeformis |
| | | | | A. madagascariensis |
| | Sympetalae | Ebenales | Sapotaceae | Madhuca utilis |
| | | Tubiflorae | Convolvulacea | Cuscuta reflexa |
| Monocotyledoneae | | Liliiflorae | Liliceae | Anemarrhena rhizoma |
| | | | | Smilax glycyphylla |
| | | | Iridaceae | Pogoniris spp. |
| | | | | Iris pseudacorus |
| | | | | Iris dichotoma |
| | | | | Belamcanda chinensis |
| | | | | Crocus aureus |
| | | Graminales | Gramineae | Cymbopogon afronardus |

The Aphloia and Mangifera species are plants whose leaves have a high content of mangiferin. Aphloia is a fully glabrous shrub, 3 to 4 m in height, originating on the East coast of Madagascar and its neighboring islands (Reunion, Mauritius, Seychelles, Comoros). The leaves of this plant are known for their diuretic, venotonic and healing properties which have led to their use as an infusion in traditional medicine.

Mangifera, and in particular Mangifera indica, belong to the Anacardiaceae family. This genus comprises sixty-two arborescent species of which sixteen species yield fruit. The species Mangifera indica L. in particular is an evergreen, upright tree with more or less large spread growing to a height of 9 to 30 m. This species currently comprises approximately 1000 varieties. The Mangifera species producing fruit are very widespread. Mangifera indica is found in particular in Asia, Central and South America and Tropical Africa.

Traditionally, the fruit of the Mangifera is eaten by local populations who also use other parts of the tree for various uses. Mangifera indica is frequently used in particular for the treatment of local populations: infusions of the bark to treat leucorrhoea, hemorrhages and dysentery. The leaves are used in decoction for hoarseness, diabetes. The ashes of the leaves are also applied to burns.

Compounds of formula I may be obtained for example by purifying extracts of all or part of these plants using any extraction or purification process (e.g. extraction with a polar solvent such as water, an alkanol, or mixture of these solvents, subsequent purification by crystallization or any other method known to those skilled in the art). Some of these methods are described for example in the patent published under number FR-A-2 486 941.

2) Chemical or Enzymatic Route.

In this connection, methods are described inter alia in the following two articles: Bhatia-V-K et al., Tetrahedron lett. (14), p. 1741-2 and Nott-P-E, Phytochemistry, vol. 6(11), p. 1597-9.

3) Biotechnological Route.

Consideration can be given to the culture of Mangifera cells in the form of calli on solid media or in fermenters, or in the form of protoplasts. But it can also be contemplated to use the bioconversion of precursors of mangiferin or its derivatives via microorganisms (e.g. yeasts, bacteria. . . ) to obtain mangiferin by extraction of these microorganisms, or by excretion in the culture medium.

Mangiferin (its isomers or its derivatives) may be used under different degrees of purity either alone or in a mixture.

It is also possible to conduct grafting, for example by esterification (by chemical or enzymatic routes) at the —OH groups of any type of molecule for example of fatty acids, short chain carboxylic acids, to obtain derivatives having different physical properties (e.g. their solubility) and/or to improve their biological activities.

Dry, purified mangiferin is on the form of yellow prismatic needles. It has no smell or taste.

It is scarcely soluble in cold water, soluble in alkaline liquids. Its solubilization is due to the formation of salts by phenol <<OH >> groups. Mangiferin (its isomers or derivatives) may be used in free form or in the form of its salts.

Cosmetic applications of mangiferin, its isomers and derivatives, in particular to protect the skin against ultraviolet radiation, have been described in patent application WO 96/16632.

The inventors have now evidenced that mangiferin and its isomers and derivatives, in purified form or contained in plant extracts, (especially in leaf extracts of Aphloia or Mangifera), increase the expression of HSPs (<<Heat-Shock Proteins >>), and inhibit the expression of MMPs (Matrix Metalloproteases) improving cell response to heat stress.

Heat stress may be the consequence in particular of sudden variation in external temperature (i.e. outside the body), or prolonged exposure to an outside temperature that is too high or too low, namely higher than 30-35° C., or lower than 10° C., respectively.

One major degradation factor for the skin is the cold (below 15° C. or 10° C.). It hardens, damages, dehydrates the skin, more than dryness alone. It acts both on the surface layers and on the deep layers. Both keratinocytes and fibroblasts take part in the phenomenon. Cold modifies the structure of the molecules (proteins, lipids) of the stratum corneum, leads to loss of viscoelastic properties and the barrier function of the skin, accelerating water loss and disorganizing molecular structure. It also induces vasoconstriction of the blood capillaries. The result is a reduced blood flow and hence a reduced supply of nutrients, vitamins, minerals, hormones, water and oxygen in the deepest skin layers.

Hot heat stress is the total calorific load exerted on the body. It arises from the metabolic production of heat on effort, and from heat brought by outside sources (air temperature, relative humidity, air circulation, sun rays and radiation from surfaces/hot materials and the insulation provided by clothing). For most people, the comfortable temperature range lies between 20° C. and 27° C., for a humidity range of between 35 and 60%. Outside these ranges, there is a feeling of discomfort. As long as the body is able to react and to adapt to ambient conditions of heat and humidity, it does not undergo the consequences. If not, the mechanisms of body heat regulation may be overtaxed, leading to disorders of greater or lesser seriousness.

As the skin forms the interface between the inside and outside of the body, it is thereby the first to be exposed to this stress. When ambient temperature increases, body temperature also tends to increase. The body reacts to maintain its inner temperature constant by increasing the blood rate of the skin and activating the sweat glands. In this way it increases the transfer of heat towards the outer surroundings to offset the supply of ambient heat.

The skin cells have complete protection systems against heat stress. Mention may be made inter alia of the antioxidizing enzymes, DNA repair enzymes and so-called heat shock proteins (HSPs).

HSPs represent a highly conserved family of proteins, naturally present in all cells of living bodies (man, animal, plants, bacteria). They are chiefly synthesized in response to a temperature increase (hence the name of <<Heat Shock Proteins>> (HSPs)).

Generally speaking, HSPs appear to recognize hydrophobic regions normally embedded within the protein, but which become accessible in proteins that have not yet been folded or denatured. HSPs are able to control the folding (conformation) of other proteins, which is called a “chaperone” function, under normal conditions in the absence of any stress factor. After exposure to a stress factor the HSPs prevent, protect or correct the denaturation, mutation and aggregation of the proteins. If the protein is too denatured, the chaperone action may give way to a protease action: when the protein is irreparable it is degraded and eliminated.

Nearly thirty HSPs have been discovered in recent years. These proteins are classified in relation to their molecular weight. They are divided into four categories: HSP90, HSP70, HSP60 and small HSPs. In the HSP70 category, the HSPs have a molecular weight close to 70 kDa.

Schematically, the family of HSPs can be divided into 2 sub-families, namely “constitutive HSPs” and “stress inducible HSPs”. Constitutive HSPs are responsible for the conformation of newly synthesized proteins, protein transport and activation of their function. The synthesis of constitutive HSPs changes with age and generally tends towards a lowering in the tissues. Inducible HSPs, as their name indicates, show an increase in their content after stress. However, with age, it is ascertained that the response time increases.

On ageing, the adaptive response to stress is highly deteriorated. HSP basic expression and induction are less effective.

HSP70s express themselves at a low level in non-stressed cells. The increase in the expression of HSP70s may be considered as a means for fighting against heat stress.

Together with this action on the expression of HSP proteins, there is an inhibiting action on matrix metalloproteases, which are secreted by human keratinocytes but also by fibroblasts. MMPs are the enzymes responsible for degradations of matrix macromolecules in the dermis and at the dermal-epidermal junction. In particular, MMP enzymes are involved in the remodeling of the dermal extracellular matrix during which their main functions are directed towards degrading the matrix components, causing detachment of the cells from their support and the release of inflammation factors, under physiological and/or pathological conditions. They are expressed in the event of stress and increasingly more so on ageing. MMP1, or collagenase-1, acts at the deep dermis and degrades collagens I and III. MMP2, or gelatinase A, and MMP9 or gelatinase B, degrade collagens I, III and VII, as well as elastin at the dermal-epidermal junction. MMP3, or stromelysin 1, degrades type IV, V collagens inter alia, as well as laminin and elastin.

The inhibition of MMP induction in response to a heat stress, by mangiferin or its derivatives, also acts in favor of preventing or reducing the effects on the skin, lips and hair caused by temperature variation (heat stress).

Consequently, the subject-matter of the invention is the cosmetic use of Mangiferin or of one of its derivatives for preventing or reducing the effects of a temperature variation on the skin, lips and hair. More precisely, the invention relates to a cosmetic treatment method for preventing or reducing the effects on the skin, lips and hair caused by a variation in temperature, which comprises the topical administration of a compound having the following general formula I:

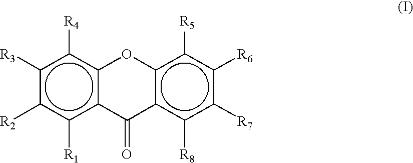

wherein each of R₁, R₂, R₃, R₄, R₅, R₆, R₇, R₈ is selected from the group consisting of —H, —OH and a glucosyl radical,

or of one of the esters or its pharmaceutically acceptable salts.

Preferably, the invention concerns the use of a formula (I) compound in which R₁, R₂, R₃, R₄, R₅, R₆, R₇, R₈ are chosen from among —H, —OH, —OCH₃ and a glucosyl radical, or one of its pharmaceutically acceptable salts.

Further preferably, at least one of R₁, R₂, R₃, R₄, R₅, R₆, R₇, R₈ is a glucosyl radical.

The compound of formula I is preferably such that R₁, R₃, R₆, R₇ are chosen from among —H, —OH, —OCH₃ and a glucosyl radical and R₂═R₅═R₈═H and R₄ is a glucosyl radical,

or R₂ is a glucosyl radical and R₄═R₅═R₈═H.

Further preferably, R₁═R₃═R₆═R₇═OH and R₂ is a glucosyl radical and R₄═R₅═R₈═H.

The selected compound may be used in the form of compositions containing this compound in proportions ranging from 0.001% to 10%, and further preferably from 0.01% to 1%, by weight of this compound with respect to the total weight of the composition.

According to one advantageous embodiment, the compound of formula I is in the form of a plant extract, preferably a leaf extract of Aphloia or Mangifera, in particular extracts obtained by extraction with polar solvents.

The extract of Aphloia or Mangifera may be used in compositions containing the same to a proportion of 0.1to 20% by weight of dry extract with respect to the total weight of the composition. The proportion of extract may however each 50% for a liquid extract.

The extract of Aphloia may be obtained from all the sub-species of Aphloia, for example Aphloia theaformis (Vahl) Benn, and Aphloia madagascariensis Clos.

The main constituents, currently identified, in the leaf extract of Aphloia according to the invention are:
a C-glucosyltetrahydroxyxanthane (Aphloiol);
flavonoids,
tannins,
three glucoside terpenes (glucoside ester of tormentic acid and of 23-hydroxytormentic acid, and glucoside ester of 6-β-hydroxytormentic acid).

It is free of alkaloid substances.

The extracts of Aphloia may be obtained by subjecting fresh, dry Aphloia leaves to extraction with a polar solvent, in particular a solvent such as water, an alkanol (e.g. ethanol, methanol), propylene glycol, butylene glycol or a mixture of these solvents. It is possible for example to use an alkanol/water mixture or a propylene glycol/water mixture.

The weight of solvent used is preferably 2 to 20 times the weight of the leaves with respect to dry weight. Advantageously, the extraction is conducted under agitation at a temperature between 10° C. and the boiling point of the solvent. Extraction time is preferably 15 minutes to 5 hours.

The extractive solutions may optionally be concentrated, and the concentrates optionally dried using means known to those skilled in the art (vacuum oven, microwave . . . ).

An example of Aphloia extract is obtained as follows:

6% (w/w) of dry Aphloia theaformis leaves of Madagascan origin, roughly crushed, are contacted under agitation and at 55° C. with a propylene glycol/water mixture (40/60). Extraction is conducted for 1 H 30. The plant is separated by filtering through a cloth (55 μm). The extract is then filtered through a disc filter (5 μm).

The pH of the extract is in the region of 5.5 and its dry extract between 0.8 and 1.3%.

Mangifera extract may be obtained from all Mangifera species, and in particular Mangifera indica L.

The chief constituents, currently identified in the leaf extract of Mangifera sp. of the invention are:
a C-glucosyltetrahydroxanthane;
flavonods,
tannins.

It is free of alkaloid substances.

The Mangifera extract is obtained in the same manner as the Aphloia extract, by subjecting the fresh, dry leaves to extraction with a polar solvent or a mixture of polar solvents (alkanol/water or propylene/water for example). These extracts may be used in liquid form, of greater or lesser concentration, or in dry form.

An example of Mangifera extract may be obtained in the following manner:

5% (w/w) of Mangifera indica dry leaves, roughly crushed, are contacted under agitation and at 50° C. with an ethanol/water mixture (30/70). Extraction is conducted for 1 H 30. The plant is separated by filtering through a cloth (55 μm). The extract is then filtered through a cellulose filter (5 μm).

Formula I compounds or plant extracts containing the same may be formulated, in association with cosmetically acceptable excipients, in cosmetic compositions, for example in the form of simple oil-in-water or water-in-oil emulsions, multiple emulsions or micro-emulsions, aqueous gels, hydroalcohols, oils, aqueous or hydroalcoholic lotions, cream, sticks, shampoo or conditioner.

These cosmetic compositions may be applied to the skin, lips or hair.

### EXAMPLES

Preparation of Mangiferin:

Mangiferin (and its derivatives) may be obtained for example by purifying extracts of Mangifera or Aphloia, using any extraction and purification method such as described above (e.g. extraction with a polar solvent such as water, alkanol, or a mixture of these solvents, then purification by crystallization or any other method known to persons skilled in the art).

Mangiferin may be used in different degrees of purity, either alone or in a mixture.

The mangiferin used in the following examples is approximately 98% pure.

### Example 1

Preventive Protection by Mangiferin of Normal Human Dermal Fibroblasts Subjected to Different Stresses.

A. Protocol

Normal human dermal fibroblasts (NHDFs) are seeded (passage 3) onto glass Labtek culture chambers (8 chambers/slide) to a proportion of 10 000 cells/chamber and incubated for 10 hours in an incubator at 37° C., 5% CO₂ and 95% humidity. The culture medium consists of DMEM devoid of phenol red (Invitrogen, ref. 31053028) supplemented with 10% fetal calf serum (FCS) (Invitrogen, ref 16010159, batch 40F1420D), 1% of a solution of non-essential amino acids (Invitrogen, ref 11140035), 100 μg/ml penicillin, 100 U/ml streptomycin, 2 mM L-gluthamine and 1% of a sodium pyruvate solution (Invitrogen, ref. 11360039).

The NHDFs are treated for 15 hours with mangiferin solubilized at 0.1% in DMSO, then diluted in the culture medium (DMEM with 1% FCS) to 2.10⁻⁴%. A reference culture medium is also made on the same slide. To validate the study, a slide is treated in the same manner but the cells are not subjected to stress.

The cells are subsequently rinsed in Hanks Balanced Saline Solution (HBSS buffer Invitrogen, ref. 14025050) then placed under hot thermal stress conditions: incubation 1 hour at +45° C. in the HBSS solution (0.4 ml per chamber.).

After the stress, the cells are rinsed in the HBSS solution then incubated 8 hours in the culture medium (DMEM with 1% FCS), in an incubator at 37° C., 5% CO₂ and 95% humidity.

The cells are rinsed in HBSS solution 8 hours after the stress. The culture chambers are removed and the cells left to dry under a laminar flow hood.

The cells are then permeabilised for 15 minutes in a formaldehyde solution (Sigma, F1635) diluted to 3.7% in HBSS. They are subsequently rinsed in an HBSS solution and fixed for 15 minutes with an iced acetone solution at −20° C. Finally, the cells are rinsed in HBSS solution and dried at ambient temperature.

For immunolabeling the cells are incubated for 1 hour with a 5% solution of PBS (Phosphate Buffered Saline)—BSA (Bovine Serum Albumin) (Sigma, A2153), to saturate the non-specific sites.

The cells are then rinsed in a 1% PBS-BSA solution for 5 minutes, and incubated overnight at +4° C. with the first anti HSP70 antibody (Stressgen, SPA812, batch B310438, rabbit polyclonal serum) diluted to 1/150 in the 1% PBS-BSA solution.

The cells are then rinsed in the 1% PBS-BSA solution for 5 minutes, and incubated in the dark for 2 hours with the second, rhodamine-conjugated, anti-rabbit antibody (Rockland, 611-1002, batch 2923) diluted to 1/500 in the 1% PBS-BSA solution.

The cells are then rinsed in the PBS solution for 5 minutes and mounted between slide and coverglass with the fluorescent mounting medium (Dako, S3023) and immunolabeling is observed under a fluorescent reverse phase microscope (Olympus, IX50).

B. Results

By implementing the above protocol, the following results have been obtained.

Labeling of the HSP70s appears to be identical, whether or not the NHDFs are treated with mangiferin, without any stress. The basic expression of the HSP70s is therefore not modified by the treatment with mangiferin, the cytoplasmic quantity appears equivalent. Mangiferin does not induce a permanent stress condition under these conditions.

On the other hand, 8 hours after the stress and without treatment, the stress proteins start to show their presence, labeling appears more intensive. The response to stress is being set up.

The labeling is further intensified when the cells are treated with mangiferin (2.10⁻⁴%) before stress. Labeling is not only more intensive at cytoplasmic level, but it is also seen at the cell core.

Mangiferin therefore enables activation of the HSP70 response solely under conditions of stress.

### Example 2

Curative Protection with Mangiferin of Normal Human Dermal Fibroblasts Subjected to Heat Stress

A. Protocol

The fibroblasts (NHDFS) are seeded (passage 3) onto glass Labtek culture chambers (8 chambers/slide) to a proportion of 10 000 cells/chamber and incubated for 10 hours in an incubator at 37° C., 5% CO₂ and 95% humidity. The culture medium consists of DMEM devoid of phenol red (Invitrogen, ref. 31053028) supplemented with 10% fetal calf serum (FCS) (Invitrogen, ref 16010159, batch 40F1420D), 1% of a solution of non-essential amino acids (Invitrogen, ref 11140035), 100 μg/ml penicillin, 100 U/ml streptomycin, 2 mM L-gluthamine and 1% of a sodium pyruvate solution (Invitrogen, ref. 11360039).

The NHDFs are rinsed in the HBSS solution then incubated 15 hours in the culture medium (DMEM with 1% FCS), in an incubator at 37° C., 5% CO₂ and 95% humidity.

The cells are subsequently rinsed in an HBSS solution (Invitrogen, ref. 14025050) then placed under conditions of hot thermal stress: incubation 1 hour at +45° C. in the HBSS solution (0.4 ml per chamber).

After the stress, the cells are rinsed in the HBSS solution then treated for 8 hours with the mangiferin solubilized in the DMSO, then diluted in the culture medium (DMEM with 1% FCS) to _{2.10}⁻⁴%. A reference culture medium is also made on the same slide. To validate the study, a slide is treated in the same manner but the cells do not undergo the stress.

The cells are rinsed in an HBSS solution 8 hours after the stress. The culture chambers are removed and the cells left to dry under a laminar flow hood.

The cells are then permeabilised for 15 minutes with a formaldehyde solution (Sigma, F1635) diluted to 3.7% in HBSS. The cells are subsequently rinsed in a HBSS solution, fixed for 15 minutes with an iced acetone solution at −20° C. Finally, the cells are rinsed in an HBSS solution and dried at room temperature.

For immunolabeling, the cells are incubated for 1 hour with a 5% PBS-BSA solution (Sigma, A2153), to saturate the non-specific sites.

The cells are then rinsed in a 1% PBS-BSA solution for 5 minutes, and incubated overnight at +4° C. with the first anti HSP70 antibody (Stressgen, SPA812, batch B310438, rabbit polyclonal serum) diluted to 1/150 in the 1% PBS-BSA solution.

The cells are subsequently rinsed in the 1% PBS-BSA solution for 5 minutes, then incubated in the dark for 2 hours with the second, rhodamine-conjugated, anti-rabbit antibody (Rockland, 611-1002, batch 2923) diluted to 1/500 in the 1% PBS-BSA solution.

Afterwards, the cells are rinsed in the PBS solution for 5 minutes and mounted between slide and coverslip with the fluorescent mounting medium (Dako, S3023) and the immunolabelings are observed under a fluorescent reverse phase microscope (Olympus, IX50).

B. Results

By implementing the above protocol, the following results have been obtained.

Labeling of the HSP70s appears identical, whether or not the NHDFs are treated with mangiferin, without stress. The basic expression of the HSP70s is therefore not modified by treatment with mangiferin, the cytoplasmic quantity appears equivalent. Mangiferin does not therefore induce a permanent stress condition under these conditions.

On the other hand, 8 hours after the stress and without treatment, the onset of the stress proteins is observed, labeling becomes more intensive. The stress response is initiated.

Labeling is then reduced when the cells are treated with 2.10⁻⁴% mangiferin after the stress.

Mangiferin, under curative conditions, therefore makes it possible to reduce the HSP70 response solely under stress conditions. Mangiferin enables a quicker return to base conditions in curative treatment, whereas the response is globally quicker and/or stronger under preventive conditions.

### Example 3

Assay of the MMP1 Metalloprotease After Heat Stimulation in the Presence of Mangiferin.

In this example, to illustrate the inhibitive action of mangiferin on the expression of MMP enzymes, the quantity of MMP1 enzymes present in normal human dermal fibroblasts (NHDFs) after a heat stress in the presence of mangiferin was compared with the quantity obtained for NHDFs in a reference medium subjected to the same conditions but without the presence of mangiferin.

This comparison was made for different conditions of heat treatment (no stress, hot stress, cold stress).

In each case, the tested NHDFs were placed in culture then treated under the following conditions:

The first day (day D), the NHDFs were seeded to a proportion of 160 000 cells/well/mL in 24-well plates in a normal medium.

The following day (day D+1), the culture medium was depleted of serum.

On day D+3, the cells were subjected to the heat conditions set forth in the table below.

When the test was conducted in the presence of mangiferin, the treatment with mangiferin was complete, i.e. the mangiferin was added to the medium to a content of 2.10⁻⁴ g/100 mL (2.10⁻⁴% w/v) 24 hours before the heat stress, during the heat stress and 24 hours after the onset of heat stress.

On the contrary, no mangiferin at all was added to the references.

After these steps, 24 hours after the heat stress, the MMP1 present in the medium was assayed using a quantitative technique of ELISA type, with a commercial Amersham kit. In parallel, the number of viable cells in the medium was determined using the MTT colorimetric test. On the basis of these elements, a MMP1 content was determined per viable cell unit.

For each of the heat treatment conditions, a reduction in MMP1 content was observed per viable cell unit in the presence of mangiferin, compared with the content obtained in the references without mangiferin. In this respect, a percentage inhibition of MMP1 expression (Pᵢ) is defined which is calculated using the following formula:
Pᵢ(as %)=(Tₜ−Tₘ)/Tₜ
in which:
Tₘ designates the MMP1 content per viable cell unit in the presence of mangiferin;
Tₜ designates the MMP1 content per viable cell unit for the reference without mangiferin under the same heat treatment conditions.

The percentage inhibition values, Pᵢ, obtained for the different heat treatment conditions are given in table II below.

TABLE II
Inhibition of the expression of MMP1 metalloprotease
in the presence of mangiferin under different conditions
of heat stress (comparison with a reference without
mangiferin under the same heat conditions).

| Heat stress conditions | Percentage inhibition Pᵢ |
|---|---|
| No heat stress: | |
| 1 h at +37° C. | 34% |
| Hot heat stress: | |
| 30 min at +45° C. | 51% |
| 1 h at +45° C. | 36% |
| 30 min at +50° C. | 40% |
| Cold heat stress | |
| 10 min at −20° C. | 22% |

The above results show that in the presence of mangiferin the dermal cells express less MMP1 after cold or hot heat stress, but also without stress (limitation of basal level). Treatment with mangiferin therefore provides effective protection against the production of these enzymes, deleterious for the conjunctive tissue, and over the longer term against skin ageing.

1. Cosmetic treatment method for preventing or reducing the effects on the skin, lips and hair caused by a variation in temperature, which comprises the topical administration of a compound having the following general formula I: 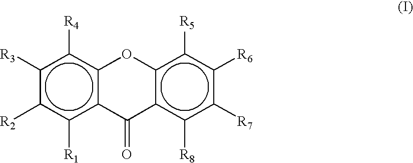
wherein each of R₁, R₂, R₃, R₄, R₅, R₆, R₇, R₈ is selected from the group consisting of —H, —OH and a glucosyl radical,
or of one of the esters or its pharmaceutically acceptable salts.

2. The method of claim 1, comprising the administration of a compound of formula I, wherein each of R₁, R₂, R₃, R₄, R₅, R₆, R₇, R₈ is selected from the group selected from —H, —OH, —OCH₃ and a glucosyl radical, or one of its pharmaceutically acceptable salts.

3. The method of claim 1 comprising the administration of a compound of formula I, wherein:
R1, R3, R6, R7 are chosen from among —H, —OH, —OCH3 and a glucosyl radical;
R2=R5=R8=H; and
R4 is a glucosyl radical,
or one of its pharmaceutically acceptable salts.

4. The method of claim 1 comprising the administration of a compound of formula I, wherein:
R₁, R₃, R₆, R₇ are chosen from among —H, —OH, —OCH₃ and a glucosyl radical;
R₂ is a glucosyl radical; and
R₄═R₅═R₈═H,
or one of its pharmaceutically acceptable salts.

5. The method of claim 4, wherein:
R₁═R₃═R₆═R₇═OH;
R₂ is a glucosyl radical;
R₄═R₅═R₈═H.

6. The method of claim 1, wherein a compound of formula (I) is used,or one of its salt or its ester, in a composition containing the same in a quantity ranging from 0.001 to 10% by weight.

7. The method of claim 6, wherein the composition contains the formula (I) compound in a quantity of 0.01 to 1% by weight.

8. The method of claim 1, wherein the compound of formula (I), or its salt or its ester, is used in the form of a plant extract.

9. The method of claim 8, wherein the plant extract is an extract of Aphloia leaves.

10. The method of claim 9, wherein the plant extract is an extract of Mangifera leaves.

11. The method of claim 8, wherein the plant extract is used in a composition containing said extract with a content of 0.01% to 20% by weight of leaf extract, expressed in weight of dry extract with respect to the total weight of the composition.

12. The method of claim 8, wherein the plant extract is obtained by extraction with a polar solvent, or by extraction with a mixture of polar solvents.
